(19) United States
(12) Patent Application Publication
(10) Pub. No.: US 2022/0054029 A1
(43) Pub. Date: Feb. 24, 2022

(54) Pulse Wave Velocity Measurement

(71) Applicant: Foundry Innovation & Research 1, Ltd., Dublin (IE)

(72) Inventors: Fiachra M. Sweeney, Dublin (IE); Orestis Vardoulis, San Francisco, CA (US)

(21) Appl. No.: 17/415,283

(22) PCT Filed: Dec. 16, 2019
(86) PCT No.: PCT/US19/66589

§ 371 Date: Jun. 17, 2021

Related U.S. Application Data
(60) Provisional application No. 62/780,456, filed on Dec. 17, 2018.

(51) Int. Cl. A61B 5/021; A61B 5/00; A61B 5/107

(52) U.S. Cl. CPC A61B 5/02125; A61B 5/7225; A61B 2562/043; A61B 5/1076; A61B 5/6876

(57) ABSTRACT

Systems are provided for determining the pulse wave velocity of blood flowing within a blood vessel. Disclosed systems may include first and second sensors at spaced apart locations in the blood vessel. The first sensor, in a first position, x1, in the vessel, r is configured to obtain a first area measurement, m1, of the vessel. The second sensor, in a second position, x2, in the vessel, is configured to obtain a second area measurement, m2, of the vessel. Disclosed systems may also include a processor configured to determine the pulse wave velocity of the vessel based on the first and second area measurements.

### RELATED APPLICATION DATA

This application claims the benefit of priority of U.S. Provisional Patent Application Ser. No. 62/780,456, filed Dec. 17, 2018, and titled “Pulse Wave Velocity Measurement”, which is incorporated by reference herein in its entirety.

### FIELD OF THE DISCLOSURE

The present disclosure is directed to measurements of characteristics of blood vessels. In particular, it is directed to estimation of vascular stiffness using pulse wave velocity (PWV) measurements background.

Heart failure, also often referred to as congestive heart failure, can occur when the myocardium cannot efficiently provide oxygenated blood to the vascular system. A variety of pathophysiological conditions, such as myocardial damage, diabetes mellitus, and hypertension gradually disrupt organ function and autoregulation mechanisms, leaving the heart unable to properly fill with blood and eject it into the vasculature. In parallel, heart failure can interact unfavorably with a series of complications such as heart valve problems, arrhythmias, liver damage and renal damage or failure.

Vascular stiffness has been suggested as a predictor of cardiovascular morbidity and mortality in a variety of populations including patients with end-stage renal disease, diabetes and hypertension. Further, pulse wave velocity (PWV) has been used as a means for estimating vascular stiffness. During cardiac ejection the left ventricle injects a bolus of blood into the ascending aorta generating a pulse and flow wave that traverses the cardiovascular system. In any medium, the velocity of wave propagation can be used to characterize its material properties. In the case of vascular tissue, higher pulse wave velocity is related to higher stiffness. Even though arteries and veins start out compliant, a number of factors can reduce their compliance. Factors such as age and disease can progressively reduce this compliance, while there can also be transitory changes in compliance due to the environmental factors, such as volumes and pressures, in and around the vein. PWV can be estimated by recording the time it takes for a wave to cross two points, along a uniform path of the vascular tree. The pulse wave velocity is the ratio of the path length divided by the pulse transit time (PTT).

A number of studies have been performed with respect to measurement of the PWV. Hellevik et al., Heart Vessels, vol. 13, no. 4, p 175-180, July 1998, performed studies on sheep liver veins. These were experimental measurements of explanted tissue in vitro using external ultrasound. Minten et. al., Cardiovasc. Res, vol. 17, no. 10, pp 627-632, October 1983, performed in vivo measurements of canine superior vena cava using invasive manometers. Nippa et al., J. Appl. Physiol., vol. 30, no. 4, pp 558-563, April 1971, relied on Doppler measurements between subclavian and femoral/wrist sites on the body. Yates PhD dissertation, Stanford Calif., SUDAAR 393 1969, was a further canine study which relied on invasive measurements based on artificially induced waves in the vena cavae of dogs. A system of reliable measurement of PWV in blood vessels would be an improvement on the state of the art and specifically facilitate the measurement of PWV to measure compliance and therefore manage intravascular blood volume.

### SUMMARY OF THE DISCLOSURE

The present disclosure provides a system for determining the pulse wave velocity of a blood vessel comprising: a first sensor in a first position, x1, in the vessel, the first sensor configured to obtain a first area measurement, m1, of the vessel; a second sensor in a second position, x2, in the vessel, the second sensor configured to obtain a second area measurement, m2, of the vessel; a processor configured to determine the pulse wave velocity of the vessel based on the first and second area measurements. The first position x1 may be along the centreline of the vessel. Furthermore, the second position x2, may be along the centreline of the vessel. This is advantageous as it provides for effective, minimally-invasive determination of the pulse wave velocity of a vessel. Utilizing two sensors provides for obtaining two area measurements of the vessel, for example the cross-sectional area of the vessel over time. The use of area measurements is advantageous as it provides information on the effect on the area of a vessel from a blood pressure pulse passing through the vessel. This area information can be used to derive the PWV of the vessel and thus information as to the condition and health of the vessel. The other advantage of this concept is that it facilitates the measurement of the compliance of the native tissue between the two sensors and is therefore not impacted by any change to the vessel due to the sensors themselves.

The distance between the two sensors along the centreline of the vessel may be determined by the equation: Δx=x2−x1 and the first and second area measurements may comprise area waveform measurements. This is advantageous as area waveform measurements provide for identifiable features, for example peaks, slopes and troughs, which can be used to correlate the effect of a blood pressure pulse passing through the vessel at the first sensor in the first position and the second sensor in the second position. Measuring the distance between the two sensors further provides for information to be derived as to the effect of the pulse in the region of the vessel between the two sensors.

Each of the area waveform measurements may comprise a respiratory component and a cardiac component. This is advantageous as it provides information from two physiological sources in a single measurement, i.e. information may be gathered about respiratory and cardiac function from a single area waveform measurement. These measurements can also then be used and compared to respiratory and cardiac signals obtained from other sources such as ECG, EEG, EMG or movement of the reader associated with the sensor. For example, specific points in the cardiac cycle can be determined and this information can be used to infer input information required for the PWV calculation. Alternatively, the cardiac signal via ECG contains information related to the respiratory cycle which can be compared to the respiratory signal from the sensor and the time delay used as an input into the pulse wave velocity calculations.

The processor of the system may be further configured to filter each of the area waveform measurements to remove the respiratory component, to provide a first filtered measurement, f1, and a second filtered measurement, f2. This is advantageous as it provides for isolation of the cardiac component of a given area waveform measurement. By being isolated in this manner, the cardiac component may be more readily analyzed for features which can be utilized for determination of the PWV of the vessel.

The processor of the system may be configured to detect a characteristic feature of the first filtered measurement and the second filtered measurement. Detecting a feature in this manner allows for the comparison of the effect of a pulse wave in the vessel at the first position in the vessel and the second position in the vessel. i.e, the effect of the same feature of the pulse wave may be compared at both sensor positions.

The characteristic feature of the waveform measurement may be a peak, a slope or a trough in the waveform. This is advantageous as such features can be readily identified by the processor without complex filtering or waveform analysis.

The processor of the system may be configured to determine the time of detection of the characteristic feature on the first filtered measurement, t1, and the time of detection of the same characteristic feature on the second filtered measurement, t2. The processor of the system may be configured to determine a time difference between the time of detection of the characteristic feature of the first filtered measurement and the second filtered measurement using the equation: Δt=t2−t1. This is advantageous as it provides for the determination of the travel time of a pulse wave through the vessel between the first sensor in the first position and the second sensor in the second position.

The processor of the system may be configured to determine the pulse wave velocity, PWV, using Δt and Δx. The PWV may be determined using the equation: PWV=Δx/Δt. In this manner, the PWV can be determined based on values derived from the initial area measurements of the vessel obtained from the first and second sensors in the first and second positions.

The processor may be configured to determine the compliance, C, of the vessel using the determined PWV and the Equation: C=V/ρ·PWV². This is advantageous as the compliance of the vessel may be determined without the requirement to obtain further measurements from the vessel. The same initial area measurements obtained from the first and second sensors in the first and second positions thus provide sufficient information to derive both the PWV and the compliance of a given vessel.

The processor of the system may be configured to determine the pressure in the vessel using the determined PWV and the determined compliance, C.

The pressure P in the vessel may be determined using the equation:

$$P = {P}_{m} + \frac{{P}_{w} + {b}_{1}}{\frac{C}{{C}_{r e f}} - {a}_{1}} ,$$

where Pₘ, P_{w}, b₁, a₁ are constants with values 20 mmHg, 30 mmHg, 5, and 0.4 respectively and C_{ref} is the vessel compliance determined with C=V/ρ·PWV² at a reference pressure of 100 mmHg. Once again, this is advantageous as the pressure within the vessel may be determined without the requirement to obtain further measurements from the vessel. The same initial area measurements obtained from the first and second sensors in the first and second positions thus provide sufficient information to derive the pressure within a given vessel.

The present disclosure provides a system for determining the pulse wave velocity of a blood vessel comprising: a first sensor in a first position, x1, on the skin overlying the vessel, the first sensor configured to obtain a first measurement, m1, of the vessel; a second sensor in a second position, x2, on the skin overlying the vessel, the second sensor configured to obtain a second measurement, m2, of the vessel; a processor configured to determine the pulse wave velocity of the vessel based on the first and second area measurements.

This is advantageous as it provides for effective and non-invasive determination of the pulse wave velocity of a vessel.

The system may comprise an array of sensors on the skin overlying the vessel, the sensors positioned along a length of the vessel, the array comprising at least one of the first sensor and the second sensor. This is advantageous as the array provides that measurements may be obtained from a number of points along the length of the vessel via a single array. Furthermore, the first and second sensors are positioned at opposite ends of the array. This provides that a comparison can be made between measurements taken from the maximum distance between sensors within the array.

The array of sensors may be comprised in a patch for application to the skin. This is advantageous as it provides for ease of application to the skin of a patient and further provides for a simple and efficient manner of obtaining measurements from a vessel.

An alternative embodiment of this concept would involve the use of two separate arrays, positioned at different anatomical locations, on the skin, over veins. The advantage of this is that the distance between the sensors is increased and therefore the transit time of the pulse wave is increased and thus the processing speed required in order to determine the transit time is reduced.

The first and second sensors may comprise at least one of an accelerometer, a pressure sensor, a flow sensor, a capacitive or inductive sensor, a PPG sensor or a distention sensor. This is advantageous as different sensor types are provided for obtaining different information types in a given measurement. Furthermore, a range of information can be obtained from the sensors from a single patch comprising multiple sensor types. The sensors may be utilized to determine a movement or pressure that is indicative of the jugular venous pressure (JVP). This is advantageous as it provides effective and non-invasive determination of the pressure of a vessel.

### BRIEF DESCRIPTION OF THE DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 schematically depicts a system and method for pulse wave velocity measurement of a vessel based on directly sensed vessel dimensions according to embodiments of the present disclosure.

FIG. 2 is a plot showing a comparison of the relative compliance of veins and arteries.

FIG. 3 schematically depicts an alternative embodiment of a system and method for obtaining non-invasive measurements according to the present disclosure.

FIG. 4 shows an aspect of the system according to the disclosure comprising a patch for obtaining non-invasive measurements.

FIGS. 5 and 6 are plots of data as determined in examples described herein.

FIG. 7 is a plot of area wave forms with separate cardiac and respiratory components.

FIG. 8 is a schematic plot of patient fluid volume versus response employing IVC diameter or area measurement (curves A₁ and A₂) in comparison to prior pressure-based systems (curve B) and in general relationship to IVC collapsibility index (IVC CI, curve C).

FIG. 9 schematically depicts a measurement being obtained from a patient via an embodiment of the system according to the disclosure.

FIG. 10 is a schematic diagram of an example of a sensor which can be used according to the system of the disclosure.

FIG. 11 shows an example of a sensor which can be used according to the system of the disclosure.

### DETAILED DESCRIPTION

Embodiments of the present disclosure provide apparatus, systems and methods for vascular pulse wave velocity (PMV) measurement using directly detected cardiac cycle waveform information taken from at least two monitoring locations in a given vessel in conjunction with a wave transit time between the points. Waveform information may be detected in a variety of ways in different disclosed embodiments. For example, waveform information may be collected with implanted vascular dimension sensors or skin surface pulsatile sensors. Furthermore, in some embodiments, detected waveform information may be used along with PWV measurements to derive vessel compliance, C, as well as the vessel pressure, P.

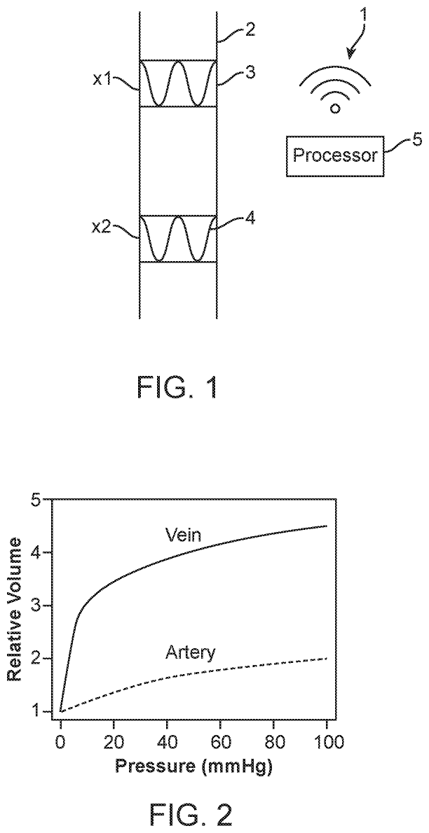

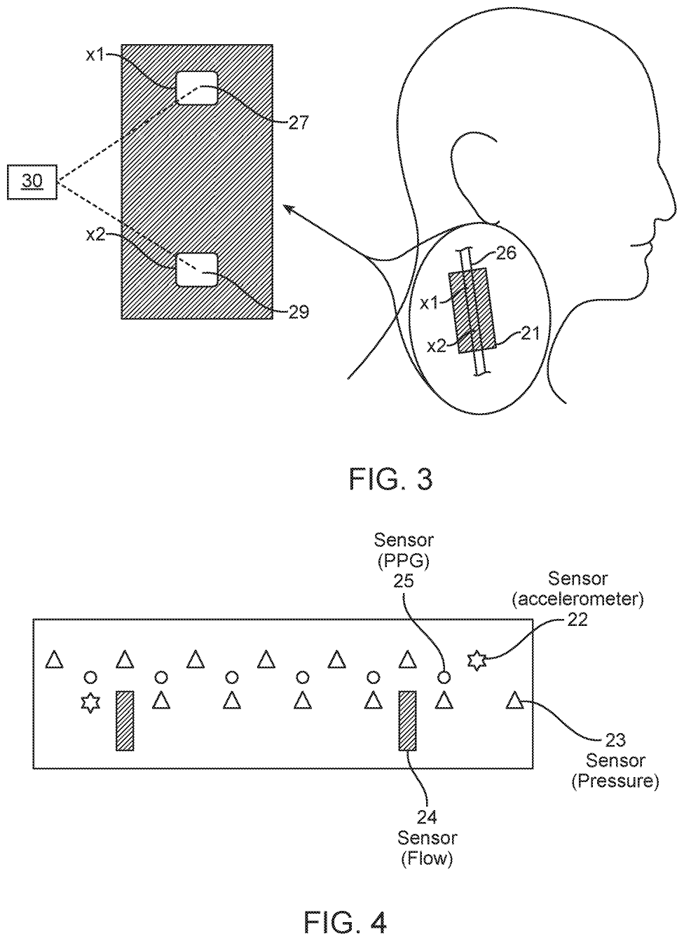

Referring generally to FIGS. 1 and 3, a system for determining pulse wave velocity of a blood vessel may comprise first and second waveform sensors (3, 4 or 27, 29) communicating with a processor (5, 30). The vascular waveform sensors are configured and dimensioned to be placed in direct contact with the patient to measure a cardiac cycle waveform at first and second positions of a blood vessel. The first and second positions are spaced apart by a known distance, which may be determined before or after sensor placement. Communicating with the sensors, the processor receives information representing the sensed waveforms and performs a number of functions using the sensed information from said sensors, including identifying at least one characteristic feature of the waveform, determining the time of travel of the characteristic feature of the waveform from the first sensor to the second sensor, and determining the pulse wave velocity of the blood vessel as ratio of the known distance between the sensors to the determined time of travel of the identified characteristic feature. The waveform information collected by the sensors may comprise waveform components attributable to other physiological mechanisms, such as for example respiration. In some embodiments, it may be desirable to further configure the processor to filter out non-cardiac related waveform components so as to focus on and specifically identify a characteristic feature of the cardiac cycle waveform.

With reference specifically to FIG. 1, an embodiment of a system 1 for determining the pulse wave velocity of a blood vessel 2 according to the present disclosure may generally comprise a first sensor 3 implanted in a first position or monitoring location, x1, in the vessel, the first sensor configured to obtain a first dimensional measurement, m1, of the vessel; a second sensor 4 implanted in a second position or monitoring location, x2, in the vessel, the second sensor configured to obtain a second dimensional measurement, m2, of the vessel and a processor 5, preferably disposed outside the patient's body, configured to determine the pulse wave velocity of the vessel based on the first and second area measurements. First and second dimensional measurements m1, m2 may comprise the area of the vessel lumen in a transverse cross-sectional plane through the vessel at positions x1 and x2, respectively. Other dimensional information such as diameter may be used. In some embodiments the first and second sensors 3, 4 are configured to produce a signal that can be received wirelessly by a receiver or reader remote from the sensors, preferably outside the patient's body.

Sensors for obtaining direct dimensional measurements of blood vessels are described further below in the section captioned “Direct Dimensional Measurements of Blood Vessels” and in more detail in Applicant's prior disclosure WO2018/031714. While certain embodiments described therein are described with respect to obtaining measurements from the IVC, the sensors described may be utilised for obtaining measurements from other vessel types, for example from the jugular vein, the superior vena cava and other vessel types. When using resonant circuit-based sensors as described therein for PWV measurement according to the present disclosure, each sensor should be tuned to different frequencies to facilitate their individual interrogation via an external reader and to avoid interference or confusion between the separate sensor output signals. One example of an external reader is a belt reader worn about the waist of an individual.

The processor 5 may take the form of a laptop or desktop computer. The processor 5 may further be a mobile telecommunication device such as a mobile telephone or tablet. The processor may further be a wearable electronic device or sensor reader. In the case that the processor is incorporated into the sensor reader, the reader shall be capable of wirelessly transmitting and receiving the required radiofrequency pulses, filtering and processing them as required and operating the appropriate software for interpreting the results. The processor is configured with suitable software for interpretation of the sensor measurements. The sensors 3, 4 and processor 5 may in some embodiments be further configured to communicate with control and communications modules, and one or more remote systems such as processing systems, user interface/displays, data storage, etc., communicating with the control and communications modules through one or more data links, preferably remote/wireless data links.

The first and second sensors 3, 4 may take the form of an implantable device. The insertion of such devices into the circulatory system of a human or animal is well known in the art and is not described in detail here. When placing sensors 3, 4 in the IVC, upper sensor 3 may be positioned inferior to the right atrium and lower sensor 4 may be positioned at or superior to the renal arteries. Alternatively, if placing sensors 3, 4 in the SVC, upper sensor 3 may be positioned within or inferior to the brachiocephalic veins and lower sensor 4 may be positioned within the SVC superior to the right atrium. To obtain measurements m1 and m2, the sensors are thus implanted into a blood vessel, with the first sensor at position x1 and the second sensor at position x2 (see FIG. 2). Once in position and activated, the sensors are capable of obtaining varying area measurements from the vessel. The processor obtains the measurements from the sensors by, for example, wireless link to or resonant coupling with the sensors. Once obtained by the processor, the measurements are processed and analyzed as set out in further detail below to determine the pulse wave velocity of the blood vessel.

Measurements of vessel diameter or area by the first and second sensor 3, 4 may be made continuously over one or more respiratory cycles to determine the variations in vessel dimensions over this cycle. Further, these measurement periods may be taken continuously, at preselected periods and/or in response to a remotely provided prompt from a signal within the system or from a healthcare provider/patient.

Determining the Pulse Wave Velocity from Vessel Dimension Measurements

Pulse wave velocity is determined in a blood vessel by identifying a pulse waveform within the vessel and then measuring the time it takes to travel between two points separated by a known distance. In the present embodiments, the known distance is set by the distance between the two sensors (Δx) and may be directly measured after implant, or, with a coordinate system, may be determined by the equation: Δx=x2−x1. This distance may be determined prior to implant, such that sensor one is specifically placed in the position x1 and the second sensor is specifically placed in the second position x2 a known distance from position x1. Alternatively, the sensors may be implanted into the vessel and Δx may be determined subsequently using a visualization technique such as ultrasound or X-ray.

The first and second dimension measurements may take the form of area waveform measurements, i.e. area measurements presented as a waveform derived from a measurement of the area of the vessel with respect to the time the measurement was obtained.

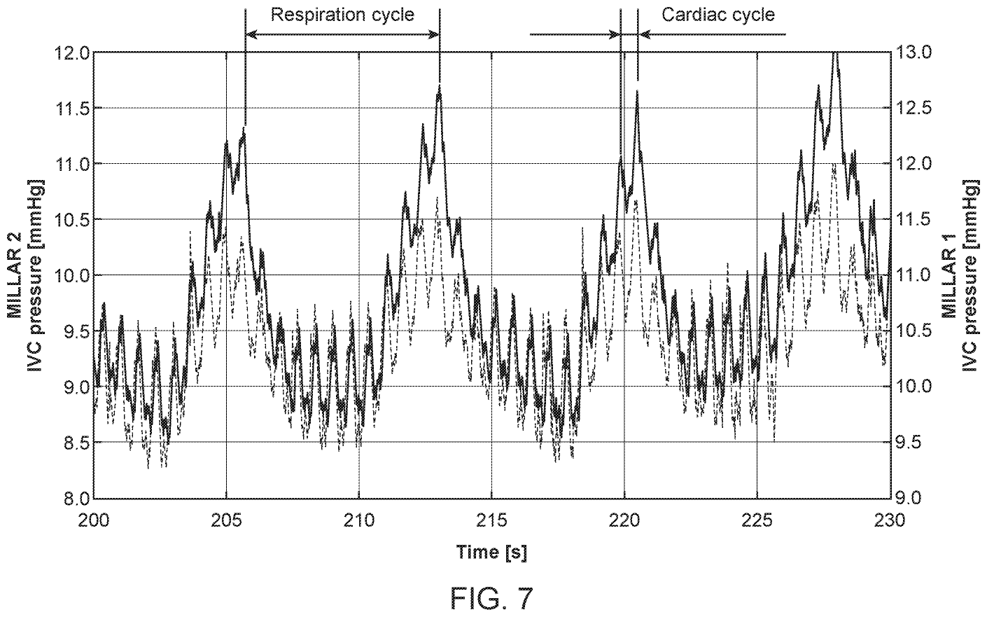

The area waveform measurements as obtained from the vessel will comprise both a respiratory component and a cardiac component, i.e. the area measurements will comprise a component resulting from both the breathing rhythm and cardiac rhythm of the subject being investigated (see FIG. 7). For measurement of the PWV, only the cardiac component is of interest. Thus, the processor of the system is further configured to filter each of the area waveform measurements to remove the respiratory component, to provide a first filtered measurement, f1 (at x1 taken at time t1), and a second filtered measurement, f2 (at x2 taken at time t2). The filtering can take place using signal processing software configured to exclude frequencies or traits associated with the respiratory component. Alternatively, a subject can be requested to hold their breath for a period of time while measurements are being obtained. Such a maneuver will also have the effect of removing the respiratory component from the sensor measurements.

Once the respiratory component of the measurements is removed, only the cardiac component should remain. The processor is thus further configured to detect a characteristic feature of the first filtered measurement at time t1, and the same characteristic feature of the second filtered measurement at time t2. The characteristic feature of the waveform measurement is typically a peak, slope or trough in the waveform, however any characteristic feature can be selected. Ideally, the feature should be readily recognizable in the waveform so as to facilitate identification and determination of the time differential based thereon. The processor is configured to then determine time differential based on the time of detection of the characteristic feature on the first filtered measurement, t1 at location x1, and the time of detection of the same characteristic feature on the second filtered measurement, t2 at location x2 using the equation: Δt=t2−t1.

Thus, the processor is further configured to obtain the PWV of the vessel from the area measurements from the sensors 3,4 by using the derived Δt value and the previously obtained Δx value using the equation: PWV=Δx/Δt. Thus, processing area measurements obtained from the implanted sensors provides a minimally invasive technique for obtaining reliable PWV values for a blood vessel.

Obtaining Compliance Values from the Obtained PWV

The obtained PWV values can be further utilized to obtain a value for the compliance, C, of the blood vessel. The relationship between pulse wave velocity and vessel compliance (change of volume for a given change in pressure) is provided analytically for a straight elastic tube via the Mons Kortweg (equation i) and its derivative the Bramwell-Hill (equation ii) equation.

The Möns Kortweg equation is defined as:

$$P W V = \sqrt{\frac{{E}_{inc} \cdot h}{2 r ρ}} \qquad ( equation i )$$

where, PWV is the pulse wave velocity, E_{inc} is the incremental elastic modulus, h is the thickness of the vessel wall, r is the radius of the vessel and ρ is the density of blood.

Bramwell & Hill proposed a series of substitutions relevant to observable hemodynamic measurements. A small rise in pressure can be shown to cause a small increase in the radius of a vessel or equally a small increase in the volume per unit length.

The Bramwell & Hill equation is defined as:

$$P W V = \sqrt{\frac{dP \cdot V}{ρ \cdot dV}} \qquad ( equation ii )$$

But with compliance C defined as change of volume as a response to a change in pressure,

$$C = \frac{dV}{d P} \qquad ( equation iii )$$

can be used to provide equation iv below:

$$P W V = \sqrt{\frac{V}{ρ \cdot C}} \qquad ( equation iv )$$

where, PWV is the pulse wave velocity, V is the volume of the vessel, p is the density of blood and C is the compliance.

As shown in the previous equations, pulse wave velocity varies with blood pressure. Vessel compliance decreases with increasing pressure due to the curvilinear relationship between venous pressure and volume. Volume increases with increased pressure, directly increasing PWV. The pressure dependency of compliance has been previously described for thoracic and abdominal aortas and has been used in the development of distributed models of the arterial tree.

As previously described, compliance can be considered as a function of a) pressure and b) location along the tree. Simply put, the compliance can be considered as a product of a pressure dependent function and a location dependent baseline value of compliance.

C(P,loc)=P_{f}(P)·C(loc,Pref) (equation v)

where, C(P, loc) is the compliance as a function of pressure and location, P_{f} (P) is pressure dependent function and C(loc, Pref) is the compliance at a specific location for a specific reference pressure (for arteries this has been previously set at 100 mmHg).

The pressure dependent function has been defined for arteries as:

$${P}_{f} ( P ) = {a}_{1} + \frac{{b}_{1}}{1 + {[ \frac{P - {P}_{max C}}{{P}_{w i d t h}} ]}^{2}} \qquad ( equation vi )$$

where, P_{f} (P) is pressure dependent function, a₁ and b₁ are constants set at 0.4 and 5 respectively, is set at 20 mmHg, P_{width} is set at 30 mmHg, and P is the pressure. Use of this function is described by Langewouters G J. in “Visco-Elasticity Of The Human Aorta In Vitro In Relation To Pressure And Age,” 1982, p. 221” and “Validation of a one-dimensional model of the systemic arterial tree” by Philippe Reymond et al. (2009).

The compliance as a function of location can be obtained via a measurement of local pulse wave velocity and the Bramwell Hill equation (as also defined above by solving for compliance).

$$C ( loc , Pref ) = \frac{V}{ρ \cdot {PWV}^{2} ( loc , Pre f )} \qquad ( equation vii )$$

The above is applicable to blood vessel types in the human body, in particular to veins and further in particular to the venae cavae, inferior vena cava (IVC) and superior vena cava (SVC). The IVC and SVC are the central vessels of the venous system. Veins are in principle much more compliant than arteries as shown in FIG. 2. Furthermore, it is important to note that venous flow is not necessarily driven by the cardiac pulse but is extensively assisted by the contraction and relaxation of surrounding skeletal and non-skeletal muscles. Knowledge of the size of the IVC and its mechanical properties has been shown to play an important role in managing treatment for fluid overload in patients with heart failure or end-stage renal disease.

Using the obtained sensor measurements from the system of the present disclosure and the above-described equations provides for extraction of a measure of the compliance of the native IVC and thus provides a highly sensitive estimate of fluid volume in the IVC as an indicator of clinical congestion. This is due to the fact that the IVC has two different compliance regions; a high compliance “normovolemic” region and a lower compliance “hypervolemic” region.

In addition, rather than solely determining the compliance of the vena cava, the compliance could further be used to compute blood flow velocity and this then used to give an estimate of volumetric flow and therefore cardiac input. This result could be used as a very close surrogate for cardiac output which is a key metric to be able to determine for continuous patient monitoring for blood volume management.

As with other applications of PWV, the pulse wave does not need to be transduced directly, it can be inferred from other biological waveforms such as ECG, EEG, EMG, etc. These can use specific points in the cardiac cycle and this information can be used to infer input information required for the pulse wave velocity calculation.

As described above, the signal from the sensors 3, 4 contains information on both the cardiac and respiratory signals. A cardiac signal (via ECG) also contains information related to the respiratory cycle. The respiratory signal from ECG can then be compared to the respiratory signal from the sensor and the time delay used as an input into the pulse wave velocity calculations.

### Obtaining Vessel Pressure

Using the previously described equations, a measure of the compliance of the vessel can be obtained. At the next step the compliance is described as a function of pressure (as defined above) and the equation is solved for pressure to yield the pressure level that is exerted on the vessel wall. In addition, or as an alternative, the trend of the pressure in the vessel can be obtained, i.e. has the pressure gone higher or lower from a baseline value. As such, the processor 5 is configured to determine the pressure in the vessel using the determined PWV and the determined compliance, C.

The pressure P in the vessel is determined using the equation:

$$P = {P}_{m} + \frac{{P}_{w} + {b}_{1}}{\frac{C}{{C}_{ref}} - {a}_{1}} ,$$

where Pₘ, P_{w}, b₁, a₁ are constants with values 20 mmHg, 30 mmHg, 5, and 0.4 respectively and C_{ref} is the vessel compliance determined with equation iv at a reference pressure of 100 mmHg.

### Non-Invasive Measurements

With reference to FIG. 3, the present disclosure further provides a system for determining the pulse wave velocity of a blood vessel comprising: a first sensor 27 in a first position, x1, on the skin overlying the vessel, the first sensor configured to obtain a first measurement, m1, of the vessel; a second sensor 29 in a second position, x2, on the skin overlying the vessel, the second sensor configured to obtain a second measurement, m2, of the vessel; a processor 30 configured to determine the pulse wave velocity of the vessel based on the first and second area measurements. Unlike the system described above, in this embodiment, the first and second sensors are placed on the skin rather than implanted into a vessel. As such, the system provides for non-invasive measurements to be obtained.

In an embodiment, the first and second sensors are formed in patch 21 which may be held on the skin via a light adhesive. An exemplary patch embodiment, as shown in FIG. 4, may comprise a number of different sensor types as sensors 27, 29, including but not limited to accelerometers 22, pressure sensors 23, flow sensors 24, PPG sensors 25 and distention sensors (not shown). Any of the different sensor types may be positioned as the first sensor in the first position x1 and as the second sensor in the second position x2. Processor 30 may be incorporated as a small chip into patch 21 or, in one alternative, sensors 27, 29 generally may include wireless transmission to communicate remotely with processor 30. In embodiments where processor 30 is incorporated into patch 21, processor 30 will include wireless transmission to communicate with a remote user interface.

In an example (FIG. 3), a non-invasive, skin-mounted patch 21 is positioned along the jugular vein 26 i.e. the line defined by the manubrio sternal joint or angle of Louis and the highest visible level of jugular vein pulsation (JVP). The patch includes an array of pulse sensing structures. The sensors can thus detect the full length of palpable vein pressure waveforms and assist in the estimation of JVP based on the current JVP measurement protocols. Furthermore, the device can capture the pulse transit time between the first sensor in the first position and the second sensor in the second position, for example, the first and second sensors may be the first and last sensors of the array. As described above, respiration components from sensor measurements may be removed computationally or via a breath hold maneuver. This leaves only the cardiac pulse generated signal in the measured waveforms. By detecting the timing of characteristic points on the pulse waveform (usually a peak, slope or a valley/trough) the pulse transit time can be estimated (the lag between the waves in the two locations) in the manner outlined above for the implanted sensors. This provides delta t. With a known distance delta x between the sensors on the array, the PWV can be obtained from the equation: PWV=Δx/Δt. Using the previously described equations, a measure of the compliance of the jugular vein can be extracted and provide a metric of vein stiffness and volume load that can be associated to a central volume overload.

### Example Data

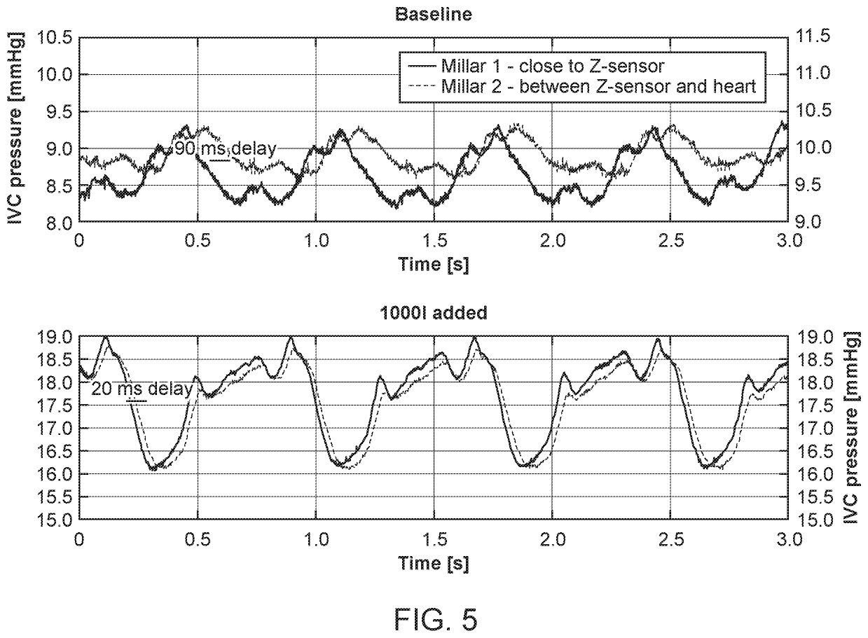

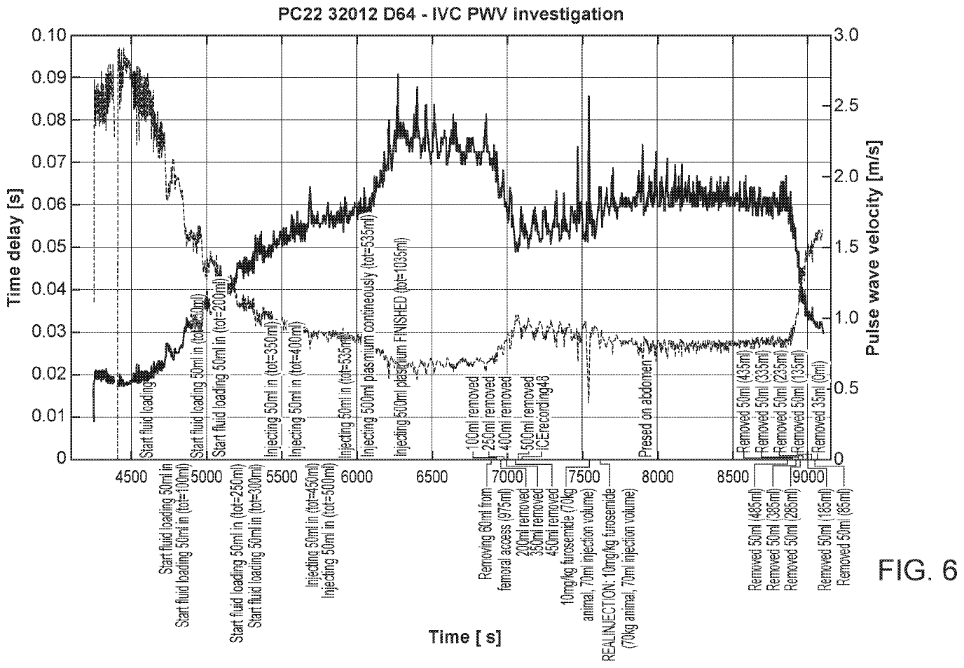

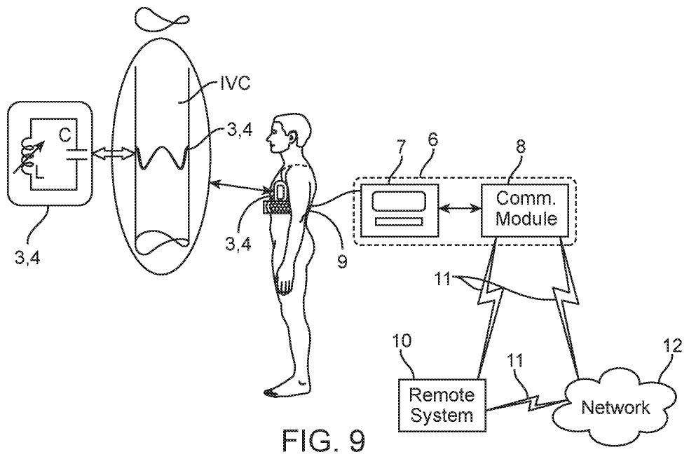

The concept described herein has been demonstrated in a preclinical model. FIGS. 5 and 6 present data from this experiment. Intravascular pressure sensors were positioned within an ovine IVC at two different locations and continuous pressure measurements recorded; one within the IVC midway between the renal arteries and the heart (FIG. 9, Millar 1) and another between this location and the heart (FIG. 9, Millar 2). Recordings were made continuously while the animal was progressively loaded with 1 litre of blood. FIG. 9 demonstrates the impact of the fluid loading on the pulse wave velocity with a delay of 90 milliseconds for the pressure wave to travel between the two sensor locations at baseline (no fluid added), and this time reducing to 20 milliseconds when 1 litre of blood is added. The changing compliance of the vessel containing the sensors changes the propagation properties of waves within it and thus changes the pulse wave velocity. FIG. 7 presents data from the same experiment demonstrating the reduction in the time delay between the sensor signals as the animal is fluid loaded, thus equating to an increase in pulse wave velocity with fluid loading. This effect is then reversed as the fluid is withdrawn from the animal.

### Direct Dimensional Measurements of Blood Vessels

As mentioned above, the assignee of the present disclosure has developed a number of devices that provide fluid volume data based on direct measurement of physical dimensions of blood vessels such as the diameter or area. Examples of these devices are described, for example, in WO2016/131020, filed Feb. 12, 2016, and WO2018/031714, filed Aug. 10, 2017, by the present Applicant, each of which is incorporated by reference herein in its entirety. Devices of the types described in these prior disclosures facilitate new management and treatment techniques based on regular intermittent (e.g., daily) or substantially continuous (near real-time), direct feedback on physical dimensions of blood vessels.

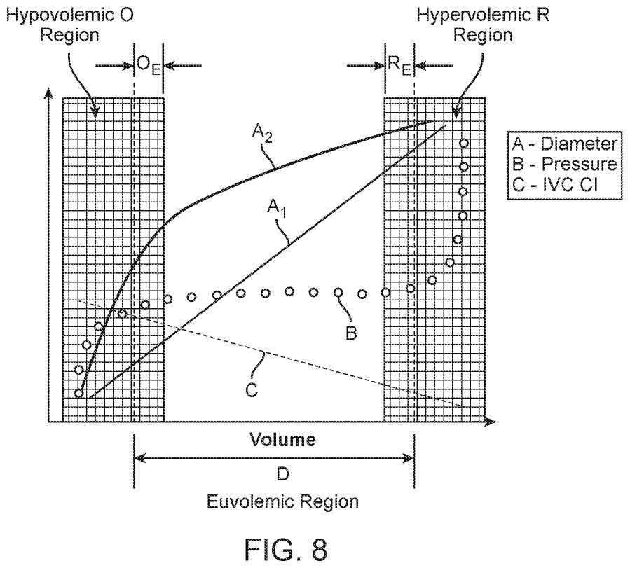

WO2018/031714 further describes some of the advantages of the information that can be derived from taking area type measurements using these devices. As can be seen in FIG. 8 (reproduced from FIG. 1 of WO2018/031714), the response of pressure-based diagnostic tools (B) over the euvolemic region (D) is relatively flat and thus provides minimal information as to exactly where patient fluid volume resides within that region. Pressure-based diagnostic tools thus tend to only indicate measurable response after the patient's fluid state has entered into the hypovolemic region (O) or the hypervolemic region (R). In contrast, a diagnostic approach based on diameter or area measurement across the respiratory and/or cardiac cycles (A₁ and A₂), which correlates directly to r C volume and IVC CI (hereinafter “IVC Volume Metrics”) provides relatively consistent sensitive information on patient fluid state across the full range of states.

It is noted in WO2018/031714 that using vessel area measurement, in this example with respect to the inferior vena cava (IVC) as an indicator of patient fluid volume, provides an opportunity for earlier response both as a sensitive hypovolemic warning and as an earlier hypervolemic warning. With respect to hypovolemia, when using pressure as a monitoring tool, a high pressure threshold can act as a potential sign of congestion, however when pressure is below a pressure threshold (i.e., along the flat part of curve B), it gives no information about the fluid status as the patient approaches hypovolemia. With respect to hypervolemia, vessel area measurements, for example, potentially provide an earlier signal than pressure-based signals due to the fact that IVC diameter or area measurements change a relatively large amount without significant change in pressure. Hence, a threshold set on IVC diameter or area measurements can give an earlier indication of hypervolemia, in advance of a pressure-based signal.

FIG. 9 shows aspects of such systems. Such a system may include a control module 6 to communicate with and, in some embodiments, power or actuate the sensor. The processor may be comprised within the control module 6. Alternatively, the processor 5 (FIG. 1) may be as a separate device. Control module 6 may include controller 7 and communications module 8. The control module may comprise a bedside console. For patient comfort, as well as repeatability in positioning, a belt reader or antenna 9 may be worn by the patient around the waist. The antenna may serve to wirelessly transmit measurements from the sensors 3, 4 to the processor 5. Information may be transferred 11 from the communications module 8 via Bluetooth, wi-fi, cellular, or local area network to a remote system 10 and/or to a network 12 for storage and/or further analysis. Further details and alternatives for control module and processor configurations are described in incorporated application WO2018/031714.

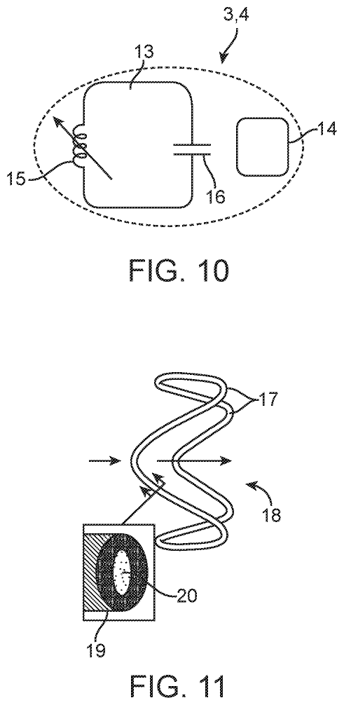

In an embodiment of the present disclosure, the first and second sensors 3, 4 may employ a variable inductance L-C circuit 13 for performing measuring or monitoring functions described herein, as shown schematically in FIG. 10. Each sensor 3, 4 may also include means 14 for securely anchoring the implant within the IVC. Using a variable inductor 15 and known capacitance 16, L-C circuit 13 produces a resonant frequency that varies as the inductance is varied. With the first sensor securely fixed at the first position, x1, and the second sensor securely fixed in a second position, x2, in the vessel, changes in shape or dimension of the vessel cause a change in configuration of the variable inductors, which in turn cause changes in the resonant frequency of the circuits. As mentioned previously, the fixed capacitors 16 of the two sensors 3 and 4, would be of different values to tune the sensors to different frequencies. The changes in the resonant frequencies can be detected and correlated to changes in the vessel shape or dimension by the processor 5 of the system.

Thus, not only should each sensor be securely positioned at their respective monitoring positions (e.g. x1/x2), but also, at least a variable coil/inductor portion 13 of the implant may have a predetermined compliance (resilience) selected and specifically configured to permit the inductor to move with changes in the vessel wall shape or dimension while maintaining its position with minimal distortion of the natural movement of the vessel wall. Thus, in some embodiments, the variable inductor is specifically configured to change shape and inductance in proportion to a change in the vessel shape or dimension.

Variable inductor 15 is configured to be remotely energized by an electric field delivered by one or more transmit coils within antenna module 9 positioned external to the patient. When energized, L-C circuit 13 produces a resonant frequency which is then detected by one or more receive coils of the antenna module. Because the resonant frequency is dependent upon the inductance of the variable inductor, changes in shape or dimension of the inductor caused by changes in shape or dimension of the vessel wall cause changes in the resonant frequency. The detected resonant frequency is then analysed by the processor component of the system to determine the vessel diameter or area, or changes therein. The vessel measurements obtained by the sensors are processed and analyzed to determine the pulse wave velocity of the blood vessel as set out in further detail below in the section titled “Determining the Pulse Wave Velocity from Area Measurements”.

An example of sensors 3, 4 for use with systems and methods described herein are shown in FIG. 11 and described further below. The sensor comprises a frame with eight crowns 17. The enlarged detail in the box of FIG. 11 represents a cross-sectional view taken as indicated. In this embodiment, sensor 18 includes multiple parallel strands of wire 19 formed around a frame 20. With multiple strands of wires, the resonant circuit may be created with either the inclusion of a discrete capacitor, element or by the inherent capacitance of the coils without the need for a separate capacitor as capacitance is provided between the wires 19 of the implant. Note that in the cross-sectional view of FIG. 11, individual ends of the very fine wires are not distinctly visible due to their small size. The wires are wrapped around frame 20 in such a way to give the appearance of layers in the drawing. Exact capacitance required for the RC circuit can be achieved by tuning of the capacitance through either or a combination of discrete capacitor selection and material selection and configuration of the wires. In one alternative sensor 18, there may be relatively few wire strands, e.g. in the range of about 15 strands, with a number of loops around the sensor in the range of about 20. In another alternative implant 18, there may be relatively more wire strands, e.g., in the range of 300 forming a single loop around the sensor.

The frame 20 may be formed from Nitinol, either as a shape set wire or laser cut shape. One advantage to a laser cut shape is that extra anchor features may cut along with the frame shape and collapse into the frame for delivery. When using a frame structure as shown in FIG. 11 the frame should be non-continuous so as to not complete an electrical loop within the implant. The coil wires may comprise fine, individually insulated wires wrapped to form a Litz wire. Factors determining inherent inductance include the number of strands and number of turns and balance of capacitance, Frequency, Q, and profile.

The words “comprises/comprising” and the words “having/including” when used herein with reference to the present disclosure are used to specify the presence of stated features, integers, steps or components but do not preclude the presence or addition of one or more other features, integers, steps, components or groups thereof.

It is appreciated that certain features of the disclosure, which are, for clarity, described in the context of separate embodiments, may also be provided in combination in a single embodiment. Conversely, various features of the disclosure which are, for brevity, described in the context of a single embodiment, may also be provided separately or in any suitable sub-combination.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

1. A system for determining the pulse wave velocity of a blood vessel, comprising:
a first sensor in a first position, x1, in the vessel, the first sensor configured to obtain a first area measurement, m1, of the vessel;
a second sensor in a second position, x2, in the vessel, the second sensor configured to obtain a second area measurement, m2, of the vessel; and
a processor configured to determine the pulse wave velocity of the vessel based on the first and second area measurements.

2. The system of claim 1, wherein the distance between the two sensors is determined by the equation: Δx=x2−x1.

3. The system of claim 2, wherein the first and second area measurements comprise area waveform measurements.

4. The system of claim 3, wherein each of the area waveform measurements comprises a respiratory component and a cardiac component.

5. The system of claim 4, wherein the processor is further configured to filter each of the area waveform measurements to remove the respiratory component, to provide a first filtered measurement, f1, and a second filtered measurement, f2.

6. The system of claim 5, wherein the processor is configured to detect a characteristic feature of the first filtered measurement and the second filtered measurement.

7. The system of claim 6, wherein the characteristic feature of the waveform measurement is a peak, slope or a trough in the waveform.

8. The system of claim 7, wherein the processor is configured to determine the time of detection of the characteristic feature on the first filtered measurement, t1, and the time of detection of the same characteristic feature on the second filtered measurement, t2.

9. The system of claim 8, wherein the processor is configured to determine a time difference for the between the time of detection of the characteristic feature of the first filtered measurement and the second filtered measurement using the equation: Δt=t2−t1.

10. The system of claim 9, wherein the processor is configured to determine the pulse wave velocity, PWV, using Δt and Δx.

11. The system of claim 10, wherein the PWV is determined using the equation: PWV=Δx/Δt.

12. The system of claim 11, wherein the processor is configured to determine the compliance, C, of the vessel using the determined PWV and the equation: C=V/ρ·PWV².

13. The system of claim 12, wherein the processor is configured to determine the pressure in the vessel using the determined PWV and the determined compliance, C.

14. The system of claim 13, wherein the pressure P in the vessel is determined using the equation:
$$P = {P}_{m} + \frac{{P}_{w} + {b}_{1}}{\frac{C}{{C}_{ref}} - {a}_{1}} ,$$
where Pₘ, P_{w}, b₁, a₁ are constants with values 20 mmHg, 30 mmHg, 5, and 0.4 respectively and C_{ref} is the vessel compliance determined with
$$P W V = \sqrt{\frac{V}{ρ \cdot C}}$$
at a reference pressure of 100 mmHg.

15. (canceled)

16. The system of claim 1, wherein the blood vessel is one of the jugular or brachiocephalic vein, the superior vena cava or the inferior vena cava, IVC.

17. A method for determining the pulse wave velocity of a blood vessel comprising:
obtaining a first area measurement, m1, of the vessel from a first sensor in a first position, x1, in the vessel;
obtaining a second area measurement, m2, of the vessel from a second sensor in a second position, x2, in the vessel;
determining the pulse wave velocity of the vessel based on the obtained first and second area measurements.

18. A system for determining the pulse wave velocity of a blood vessel, comprising:
a first sensor in a first position, x1, on the skin overlying the vessel, the first sensor configured to obtain a first measurement, m1, of the vessel;
a second sensor in a second position, x2, on the skin overlying the vessel, the second sensor configured to obtain a second measurement, m2, of the vessel;
wherein the distance between the first position and the second position is determined by the equation Δx=x2−x1, and
a processor configured to determine the pulse wave velocity of the vessel based on the first and second area measurements.

19. The system of claim 18, comprising an array of sensors on the skin overlying the vessel, the sensors positioned along a length of the vessel, the array comprising at least one of the first sensor and the second sensor.

20. The system of claim 19, wherein the first and second sensor are positioned at opposite ends of the array.

21. The system of claim 20, wherein the array of sensors is comprised in a patch for application to the skin.

22.-44. (canceled)
